(19) United States
(12) Patent Application Publication
(10) Pub. No.: US 2020/0375866 A1
(43) Pub. Date: Dec. 3, 2020

(54) HAIR TREATMENT PROCESS USING A FLAVIN DERIVATIVE AND LIGHT RADIATION

(71) Applicant: L'OREAL, Paris (FR)

(72) Inventor: Andrew GREAVES, AULNAY-SOUS-BOIS (US)

(21) Appl. No.: 16/955,003

(22) PCT Filed: Dec. 19, 2018
(86) PCT No.: PCT/EP2018/085881

§ 371 Date: Jun. 17, 2020

(30) Foreign Application Priority Data
Dec. 20, 2017 (FR) 1762600
Dec. 20, 2017 (FR) 1762638

(51) Int. Cl. A61K 8/55; A61K 8/49; A61K 8/64; A61Q 5/00

(52) U.S. Cl. CPC A61K 8/55; A61Q 5/002; A61K 8/64; A61K 8/4953

(57) ABSTRACT

The present invention relates to a hair treatment process, comprising the application of a composition comprising i) at least one flavin derivative and at least one step of exposing the hair to artificial or natural light radiation, in particular for caring for and/or repairing the hair.

The present invention relates to a hair treatment process, comprising the application of a composition comprising i) at least one flavin derivative and at least one step of exposing the hair to artificial or natural light radiation, in particular for caring for and/or repairing the hair.

The hair is damaged and weakened by external atmospheric agents such as pollution and bad weather, and also by mechanical or chemical treatments, such as brushing, combing, dyeing, bleaching, permanent-waving, relaxing and repeated washing. The hair thus becomes damaged and may in the long run become dry, coarse, brittle, dull, split and/or soft or else sensitive to humidity, making the hair unmanageable, often with frizziness, and/or difficult to style in a humid environment, in particular in a very humid environment. The hair is then also sensitive to mechanical constraints such as being gripped with an elastic means, for example to make a ponytail, and retains the gripping mark even after the elastic band has been taken off. When the hair is soft, it is difficult to style and to volumize, in particular close to the scalp. The term “flat hair” is then often used, somewhat imprecisely, to express this lack of volume close to the roots.

To overcome these drawbacks, it is common practice to make use of hair compositions for conditioning the hair by giving it cosmetic properties and also good shaping.

However, the conditioning effect obtained via these hair treatments fades out rapidly over time, and in particular does not have satisfactory persistence with respect to shampoo washing.

Furthermore, these hair compositions have little or no effect on controlling the volume of the hair and/or holding the hairstyle and/or managing the hair in a humid or even a very humid environment (humidity-resistant shape and volume control). They also have little or no effect on the sensitivity to mechanical constraints such as being gripped with an elastic band or on the lack of volume of the hair close to the roots.

There is thus a real need to develop processes for treating the hair that are capable of conserving or even improving the quality of the fibre such as the softness, disentangling, smoothness, manageability, volume and strength of the hair, for example by reducing the brittleness of the hair, and doing so in a persistent manner, or else by making the hair less sensitive to mechanical constraints such as gripping, or by allowing control of the volume and frizziness of the hair and also of the hairstyle in a humid environment, or even by giving soft hair volume close to the roots, and doing so all the more so in a manner that is resistant to humidity and to shampoo washing.

In addition, it is known that riboflavin can initiate photochemical reactions such as the crosslinking of polysaccharides to form gels (see, for example, WO 2010/083039, J. Biomed. Mater. Res. B Appl Biomater., Kim, S. H., Chu, C. C., 91(1), 390-400 (2009).

It is also known practice to use riboflavins combined with collagen and UV-A for treating the cornea of the eye (see, for example, J. Cataract Refract. Surg. Feb. 36(2): 273-6. (2010) doi: 10.1016/j.jcrs.2009.07.041.) This combination is not used in the field of haircare for repairing the hair.

WO 2016/126121 describes a hair and scalp composition comprising a yeast extract comprising vitamin B2 (riboflavin).

There is still a need to develop a hair treatment process, more particularly a process for treating the hair in an efficient and long-lasting manner while at the same time limiting the perceived degradation of the hair, in particular that is persistent with respect to successive shampoo washes and to control the shape of the hair fibres that resists humidity.

There is still a need to develop an efficient and long-lasting process for treating the hair in for limiting the perceived degradation of the hair, especially the perception of dry, coarse, brittle, dull, split and/or soft hair or alternatively hair that is humidity-sensitive often with frizziness, and/or hair that is difficult to style in a humid environment, in particular in a very humid environment, and/or for repairing damaged hair.

There is also a need to develop a hair treatment process for making the hair insensitive to mechanical constraints such as gripping with an elastic band, for example to make a ponytail, or for giving the hair volume in a long-lasting manner, which is resistant to humidity and to shampoo washing, especially close to the roots. Furthermore, there is a real need to develop a hair treatment process that withstands shampoo washing at least five times.

This (these) aims are achieved by the process of the invention, i.e. a hair treatment process, comprising:

1) a step of applying a composition (A) comprising:
i) one or more flavin derivatives chosen from the compounds of formula (I) below:

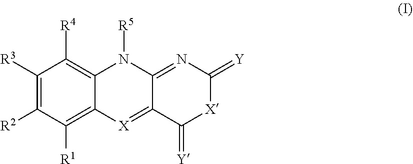

and also the organic or mineral acid or base salts thereof, the optical isomers, geometrical isomers and tautomers thereof, and the solvates thereof such as hydrates; in which formula (I):
R¹, R², R³ and R⁴, which may be identical or different, represent a hydrogen atom, a halogen atom or a group chosen from hydroxyl, (C₁-C₆)alkyl, (C₁-C₆)alkoxy, (C₁-C₆)alkylthio, (di)(C₁-C₆)(alkyl)amino, nitro(so), in particular chosen from hydrogen and (C₁-C₆)alkyl; more particularly, R¹ and R⁴ represent a hydrogen atom and R² and R³ represent a hydrogen atom or a (C₁-C₄)alkyl group such as methyl;
R⁵ represents a hydrogen atom or a (C₁-C₈)alkyl group optionally substituted with one or more groups chosen from i) hydroxyl, ii) R⁶—C(Y″)—O— with R⁶ representing a hydrogen atom or a (C₁-C₄)alkyl or aryl(C₁-C₄)alkyl group such as benzyl and Y″ representing an oxygen or sulfur atom, or N(R⁷) with R⁷ representing a hydrogen atom or a (C₁-C₄)alkyl group, Y″ preferably representing an oxygen atom, iii) phosphoric (HO)₂P(O)—O—, iv) —O—[(HO)P(O)—O]ₙ-Suc-Het with Suc representing a divalent sugar group such as ribose, and Het representing a heteroaryl group such as adenine, n being an integer equal to 0, 1 or 2, preferably 2; in particular, the group Suc-Het represents a ribose group substituted with an adenine group;
X represents a nitrogen atom or a methylene group C(R⁸) with R⁸ denoting a hydrogen atom or a (C₁-C₄)alkyl group; preferably, X represents a nitrogen atom;
X′ represents an oxygen atom or a group NR⁹ with R⁹ representing a hydrogen atom or a (C₁-C₆)alkyl group; in particular, X′ denotes NR⁹, preferably NH;
Y and Y′, which may be identical or different, represent an oxygen or sulfur atom or a group NR¹⁰ with R¹⁰ representing a hydrogen atom or a (C₁-C₆)alkyl group, preferably Y and Y′ represent an oxygen atom;
   the flavin derivative(s) being in an amount inclusively between 0.01% and 30% by weight relative to the total weight of composition (A), in particular between 0.05% and 20%, more particularly between 0.1% and 10%, preferentially between 1% and 5% by weight relative to the total weight of composition (A); and
   2) a step of supplying energy to the hair, which consists in exposing the hair to at least one artificial or natural light radiation,
   it being understood that steps 1) and 2) may be performed simultaneously with the application, or sequentially, preferably simultaneously with the application or else sequentially to the application 1) and then 2).

The invention also relates to a composition (B) comprising i) one or more compounds of formula (I) as defined previously, ii) one or more polyamino acids and iii) one or more amines i) other than the compounds of formula (I), and ii) other than the polyamino acids, preferably one or more amines chosen from the amino acids of formula (III) defined below.

A subject of the invention is also a multi-compartment kit or device comprising:
either, in one compartment, ingredient i) one or more compounds of formula (I) as defined previously and, in another compartment, ingredient ii) one or more polyamino acids, or, in one compartment, ingredients i) and ii) together, and
in another compartment an artificial-light-emitting device.

A subject of the invention is also a multi-compartment kit or device comprising:
in one compartment, i) one or more compounds of formula (I) as defined previously, and optionally in the same compartment or in a different compartment, iii) one or more amines other than i) the compounds of formula (I), and other than ii) the polyamino acids; and
in another compartment an artificial-light-emitting device.

Another subject of the invention is the cosmetic use i) of one or more compounds of formula (I) as defined previously and ii) of one or more polyamino acids and of artificial or natural light radiation; in particular for caring for and/or repairing the hair, preferably for repairing the hair.

Finally, a last subject of the invention is the cosmetic use i) of one or more compounds of formula (I) as defined previously and optionally iii) of one or more amines as defined previously, and of artificial or natural light radiation; in particular for caring for and/or repairing the hair, preferably for repairing the hair.

The process of the invention involving the application to the hair of a composition (A) containing one or more flavin derivatives chosen from the compounds of formula (I) as defined previously, said application being followed by a step of irradiation with at least one light radiation, makes it possible to improve the cosmetic quality of the hair. The hair appears repaired. Hair treated via the process of the invention has more volume, especially close to the roots, by means of a root-lifting effect that is resistant to high hygrometries and the effect being persistent with respect to shampoo washing, i.e. hair treated via the process of the invention retains its volume and its shape even after exposure to high hygrometries such as a relative hygrometry of 80%, and this transformation withstands repeated shampoo washing.

Other characteristics and advantages of the invention will emerge more clearly on reading the description and the examples that follow.

In the following text, unless indicated otherwise:
the term “salt” means the salts of addition with an organic or mineral acid or base;
the term “organic or mineral acid salt” means cosmetically acceptable addition salts obtained by addition of an organic or mineral acid, more particularly the salts chosen from a salt derived from i) hydrochloric acid HCl, ii) hydrobromic acid HBr, iii) sulfuric acid H₂SO₄, iv) alkylsulfonic acids: Alk—S(O)₂OH such as methylsulfonic acid and ethylsulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk—O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; xiv) triflic acid CF₃SO₃H; and xv) tetrafluoroboric acid HBF₄;
the term “alkyl” means a linear or branched radical containing from 1 to 12 carbon atoms, in particular from 1 to 8 carbon atoms, more particularly from 1 to 8 carbon atoms, preferably from 1 to 4 carbon atoms, for example methyl, ethyl, n-propyl, isopropyl, butyl, n-pentyl, n-hexyl, preferably methyl or n-propyl and more preferentially methyl;
the term “(Cₓ-C_{y})alkyl” means an alkyl radical as defined previously, said alkyl radical comprising x to y carbon atoms;
the term “alkylene” means a linear or branched divalent radical containing from 1 to 16 carbon atoms, in particular from 1 to 12 carbon atoms, preferably from 1 to 8 carbon atoms, more preferentially from 1 to 6 carbon atoms, even more preferentially from 1 to 4 carbon atoms, for example methylene, ethylene, n-propylene, isopropylene, butylene, n-pentylene or n-hexylene, preferably methylene;
the term “(Cₓ-C_{y})alkylene” means an alkylene radical as defined previously, said alkylene radical comprising x to y carbon atoms;
the term “alkoxy” means an alkyl-oxy group with “alkyl” as defined previously;
the term “(Cₓ-C_{y})alkoxy” means an alkoxy radical as defined previously, said alkoxy radical comprising x to y carbon atoms;
an “aryl” radical represents a monocyclic or polycyclic fused or non-fused carbon-based group, comprising from 6 to 22 carbon atoms, at least one ring of which is aromatic; in particular, the aryl radical is a phenyl, biphenyl, naphthyl, indenyl, anthracenyl or tetrahydronaphthyl, preferably phenyl;
a “heteroaryl radical” represents a 5- to 22-membered, monocyclic or polycyclic, fused or non-fused group, comprising from 1 to 6 heteroatoms chosen from nitrogen, oxygen and sulfur atoms, at least one ring of which is aromatic;

preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, pyrimidyl, pyrimidyl-one, pyrimidyl-dione, tetrazolyl, dihydrothiazolyl, imidazopyridinyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, purinyl-one, purinylepyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl;
the term “sugar” or “Suc” means a monosaccharide group or a disaccharide group, preferably a monosaccharide, each saccharide unit (the saccharide unit in the case of a monosaccharide or each saccharide unit in the case of a disaccharide) comprising one or more hydroxyl groups optionally substituted with a radical R″ chosen from: i) (C₁-C₆)alkyl, (C₂-C₆)alkenyl; ii) an acetyl radical; or iii) a protecting group (PG) for hydroxyl functions, such as (C₂-C₆)alkyl(thio)carbonyl, preferably (C₂-C₆)alkylcarbonyl; in particular R″ represents a (C₁-C₆)alkyl group such as methyl, or an acetyl group; said monosaccharide or disaccharide radical being linked to the rest of the molecule via a bond between the carbon atom C₁ of one of the sugars of said monosaccharide or disaccharide radical, this bond possibly being α or β anomeric; it is understood that, for the compounds of formula (I) as defined previously, when Suc represents a monosaccharide radical, then it is in pyranose form (the sugar heterocycle which constitutes it is 6-membered) or furanose form (the sugar heterocycle which constitutes it is 5-membered); and when Suc represents a disaccharide radical, it comprises a sequence of two identical or different saccharide or oside units which may be in furanose or pyranose form. Preferably, the disaccharide results from the sequence of a saccharide unit in furanose form and a unit in pyranose form or the sequence of a saccharide unit in pyranose form and a unit in furanose form; whether it is for the monosaccharide or polysaccharide radical, each saccharide unit may be in levorotatory L or dextrorotatory D form, and in α or β anomeric form;
the term “polyamino acids” means peptides constituted of amino acids connected together via peptide bonds, especially those of formula (III) as defined below, said peptides being of variable length ranging from oligopeptides containing from two to several tens of amino acids up to polypeptides including a larger number of amino acids and which may be derived from an mRNA translation, said peptides also possibly being assembled to constitute proteins such as collagen;
the term “polysaccharides” means a polysaccharide sugar which is a polymer constituted of several saccharides bonded together via O-oside bonds, said polymers being constituted of monosaccharide units as defined previously, said monosaccharide units comprising at least 5 carbon atoms, preferably 6; in particular, the monosaccharide units are linked together via a 1,4 or 1,6 bond as α (alpha) or β (beta) anomer, it being possible for each oside unit to be of L or D configuration, and also the salts thereof and the solvates thereof such as the hydrates of said monosaccharides; more particularly, they are polymers formed from a certain number of saccharides (or monosaccharides) having the general formula: —[Cₓ(H₂O)_{y})]ₙ— where x is an integer greater than or equal to 5, preferably x is greater than or equal to 6, in particular x is between 5 and 7 inclusive and preferably x=6, and y is an integer which represents x−1, and n is an integer greater than or equal to 2, particularly between 3 and 3000 inclusive, more particularly between 5 and 2500 and preferentially between 10 and 2300;
the term “amino monosaccharide or polysaccharide” means that the monosaccharide or polysaccharide is substituted with one or more amino groups NR₁R₂, i.e. at least one of the hydroxyl groups of at least one saccharide unit is replaced with a group NR₁R₂ with R₁ and R₂, which may be identical or different, representing i) a hydrogen atom, ii) a (C₁-C₆)alkyl group that is optionally substituted, preferably with one or more hydroxyl or NH₂ groups, iii) an aryl group such as phenyl, iv) an aryl(C₁-C₄)alkyl group such as benzyl, v) a (hetero)cyclo(C₅-C₇)alkyl group such as cyclohexyl, morpholinyl, piperazinyl, piperidinyl, vi) a (hetero)cyclo(C₅-C₇)alkyl(C₁-C₄)alkyl group such as cyclohexylmethyl, vii) —C(Y)—(Y′)ₚ—R′1 with Y and Y′, which may be identical or different, representing an oxygen atom, a sulfur atom or N(R′₂), preferably oxygen, p=0 or 1, preferably 0; and R′₁ and R′₂ representing i) to vi) of R₁ and R₂ defined previously, and in particular R′1 denoting a (C₁-C₆)alkyl group such as methyl. Preferably R₁ and R₂ represent a hydrogen atom or a (C₁-C₄)alkylcarbonyl group such as acetyl;
moreover, the addition salts that may be used in the context of the invention are especially chosen from salts of addition with a cosmetically acceptable base such as the basifying agents as defined below, for instance alkali metal or alkaline-earth metal hydroxides such as sodium hydroxide or potassium hydroxide, ammonia, amines or alkanolamines;
the term “heterocycle” means a 5- to 10-membered monocyclic or bicyclic, preferably monocyclic, fused or non-fused, saturated or unsaturated, aromatic or non-aromatic group, containing from 1 to 3 heteroatoms chosen from nitrogen, oxygen and sulfur atoms, this heterocycle possibly being substituted with one or more radicals, which may be identical or different, chosen from alkyl, hydroxyalkyl and alkoxy radicals; preferentially, according to the present invention, the saturated or unsaturated, preferably saturated, and 5- to 8-membered, heterocycle is more preferentially chosen from piperidyl, pyrrolidinyl, piperazinyl and morpholinyl;
the term “cycloalkyl” means a 5- to 10-membered monocyclic or bicyclic, preferably monocyclic, fused or non-fused hydrocarbon-based group which is saturated or contains one or more ethylenic unsaturations, and which is non-aromatic, this cycloalkyl possibly being substituted with one or more radicals, which may be identical or different, chosen from alkyl, hydroxyalkyl and alkoxy radicals; preferentially, according to the present invention, the cycloalkyl is saturated and 5- to 8-membered, and more preferentially is chosen from cyclopentyl and cyclohexyl;
the “heterocycle”, “cycloalkyl”, “aryl” and “heteroaryl” radicals may be substituted with at least one substituent borne by a carbon atom or a heteroatom, chosen from:
   an optionally substituted C₁-C₆ alkyl radical;
   a halogen atom;
   a hydroxyl group;
   a C₁-C₂ alkoxy radical;
   a (poly)hydroxy(C₂-C₄)alkoxy radical;
   an amino radical;
   a 5- or 6-membered heterocycloalkyl radical;
   a 5- or 6-membered heteroaryl radical, optionally substituted with a (C₁-C₄)alkyl radical, preferentially methyl;
   an amino radical substituted with one or two identical or different C₁-C₆ alkyl radicals, optionally bearing at least:
i) a hydroxyl group,
ii) an amino group optionally substituted with one or two optionally substituted C₁-C₃ alkyl radicals, said alkyl radicals possibly forming with the nitrogen atom to which they are attached a saturated or unsaturated, optionally substituted 5- to 7-membered heterocycle, optionally comprising at least one other nitrogen or non-nitrogen heteroatom,
   an acylamino radical (—NR—C(O)—R′) in which the radical R is a hydrogen atom or a C₁-C₄ alkyl radical optionally bearing at least one hydroxyl group and the R′ radical is a C₁-C₂ alkyl radical;
   a carbamoyl radical ((R)₂N—C(O)—) in which the radicals R, which may be identical or different, represent a hydrogen atom or a C₁-C₄ alkyl radical optionally bearing at least one hydroxyl group;
   an alkylsulfonylamino radical (R′—S(O)2—N(R)—) in which the radical R represents a hydrogen atom or a C₁-C₄ alkyl radical optionally bearing at least one hydroxyl group and the radical R′ represents a C₁-C₄ alkyl radical or a phenyl radical; an aminosulfonyl radical ((R)2N—S(O)2—) in which the radicals R, which may be identical or different, represent a hydrogen atom or a C₁-C₄ alkyl radical optionally bearing at least one hydroxyl group;
   a carboxylic radical in acid or salified (preferably with an alkali metal or a substituted or unsubstituted ammonium) form;
   a cyano group;
   a nitro or nitroso group;
   a polyhaloalkyl group, preferentially trifluoromethyl;
the term “elastic means” means any means for gripping keratin fibres, in the form of a short yarn, band or strip comprising at least one part made of elastic material such as rubber, of generally circular shape,
the expression “at least one” is equivalent to “one or more”; and
the expression “inclusive” for a range of concentrations means that the limits of the range are included in the defined interval.
   i) The flavin derivatives of formula (I)

Compositions (A) or (B) of the invention comprise i) one or more flavin derivatives chosen from the compounds of formula (I) as defined previously and also organic or mineral acid or base salts thereof, optical and geometric isomers thereof, tautomers and solvates thereof such as hydrates:

According to a particular embodiment of the invention, the compounds of formula (I) are such that R¹, R², R³ and R⁴, which may be identical or different, represent a hydrogen atom, (C₁-C₆)alkyl, (C₁-C₆)alkoxy or (di)(C₁-C₄)(alkyl)amino, in particular chosen from hydrogen and (C₁-C₄)alkyl. Preferably, R¹ and R⁴ represent a hydrogen atom, and R² and R³ represent a hydrogen atom or a (C₁-C₄)alkyl group such as methyl, preferably a (C₁-C₄)alkyl group such as methyl.

According to a particular embodiment of the invention, the compounds of formula (I) are such that R⁵ represents a hydrogen atom or a (C₁-C₆)alkyl group such as methyl.

According to another preferred embodiment of the invention, the compounds of formula (I) are such that R⁵ represents a (C₁-C₆)alkyl group substituted with one or more identical or different groups chosen from i) hydroxyl, ii) R⁶—C(O)—O— with R⁶ representing a hydrogen atom or a (C₁-C₄)alkyl group such as methyl or n-propyl; iii) phosphoric (HO)₂P(O)—O—, iv) —O—[(HO)P(O)—O]ₙ-Suc-Het with Suc representing a monosaccharide such as ribose, and Het representing an optionally substituted heteroaryl group, in particular optionally substituted pyrimidyl-dione such as adenine, n being an integer equal to 1 or 2, preferably 2; in particular, Suc-Het represents a ribose group, preferably α-D-ribose, optionally substituted with an adenine group.

According to a preferred embodiment, the compounds of formula (I) are such that the radical Suc represents a divalent monosaccharide radical in which the heterocycle constituting it contains 4 or 5 carbon atoms, having the following formula:

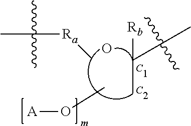

Rₐ representing a (C₁-C₄)alkylene group such as methylene and Rₐ being linked to the rest of the molecule via at least one phosphate group, it being understood that the radical Rₐ is in position C⁵ if the sugar unit is in pyranose form or in position C⁴ if it is in furanose form;
R_{b} represents a hydrogen atom or a group —CH₂—O—A; preferably, R_{b} denotes a hydrogen atom;
A represents a hydrogen atom, a (C₁-C₆)alkyl group or a hydroxy-function-protecting group, such as R_{c}—C(O)— with R_{c} representing a hydrogen atom or a (C₁-C₄)alkyl group such as methyl, or else, when n is greater than or equal to 2 and two groups A—O are contiguous, then two groups A may together form a linear or branched (C₁-C₆)alkylene chain; preferably, all the groups which protect A are identical;
m is equal to 1, 2 or 3; preferably, m=2;
[symbol] represents the two substitution parts of the divalent monosaccharide.

According to a more particular embodiment, the compounds of formula (I) are such that R⁵ represents the following group:

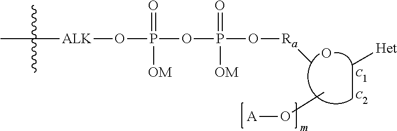

with Ra, A, Het and m as defined previously;
ALK representing a (C₁-C₆)alkylene group, preferably pentylene, optionally substituted with one or more hydroxyl groups;
M representing a hydrogen atom or a cationic counterion, in particular an alkali metal or alkaline-earth metal such as Na⁺, K⁺ or ammonium, preferably Na⁺ and
[symbol] representing the part that is linked to the rest of the molecule.

Preferably, Het represents a purine or pyrimidine nitrogenous base such as adenine, thymine, guanine, cytosine or uracil. Preferably, Het represents an optionally substituted bicyclic heteroaryl group; more particularly, Het represents a purinyl-dione group which is optionally substituted especially with one or more (di)(C₁-C₄)(alkyl)amino groups; preferentially, Het is a purine nitrogenous base such as adenine.

According to a more particular embodiment, the compounds of formula (I) are such that R⁵ represents a (C₁-C₆)alkyl group optionally substituted with one or more hydroxyl or phosphate groups such as pentyl substituted with one, two, three or four hydroxyl groups and optionally with a phosphate group, such as 2,3,4,5-tetrahydroxypentyl or 5-dihydrogen phosphate 2,3,4-trihydroxypentyl.

According to a particular embodiment, the compounds of formula (I) are such that X represents a nitrogen atom.

According to a particular embodiment, the compounds of formula (I) are such that X′ represents a group NR⁹ with R⁹ representing a hydrogen atom or a (C₁-C₆)alkyl group; in particular, X′ preferably denotes NH.

According to a particular embodiment, the compounds of formula (I) are such that Y and Y′ are identical and more particularly represent an oxygen or sulfur atom; preferably, Y and Y′ represent an oxygen atom.

More particularly, the compounds of formula (I) of the invention are chosen from compounds 1 to 7 below:

| Structure | |
|---|---|
| 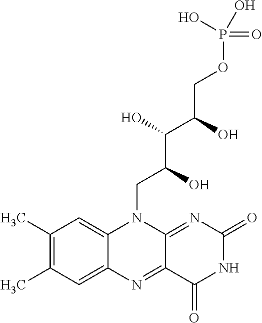 [SMILES: Cc1cc2nc3c(=O)[nH]c(=O)nc-3n(C[C@H](O)[C@H](O)[C@H](O)COP(=O)(O)O)c2cc1C] | 1 |
| (2R,3S,4S)-5-(7,8-dimethyl-2,4-dioxo-3,4-dihydrobenzo[g]pteridin- | |
| 10(2H)-yl)-2,3,4-trihydroxypentyl dihydrogen phosphate or | |
| riboflavin 5″-phosphate, or riboflavin-5′-phosphate | |
| 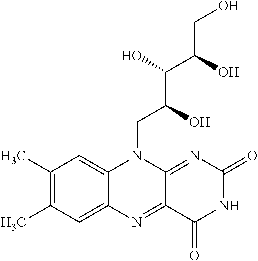 [SMILES: Cc1cc2nc3c(=O)[nH]c(=O)nc-3n(C[C@H](O)[C@H](O)[C@H](O)CO)c2cc1C] | 2 |
| 7,8-dimethyl-10-((2R,3R,4S)-2,3,4,5-tetrahydroxypentyl)benzo[g] | |
| pteridine-2,4-(3H,10H)-dione, or vitamin B₂, lactoflavin | |
| 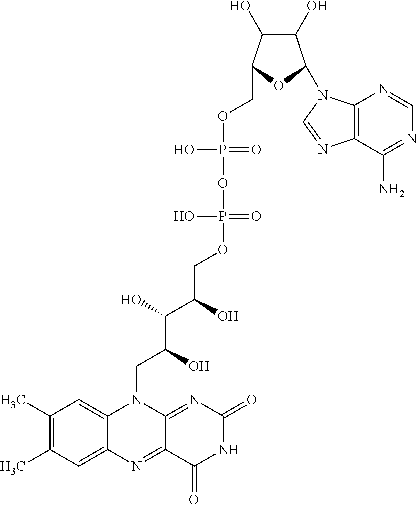 | 3 |
| Flavin adenine dinucleotide (FAD) | |
| 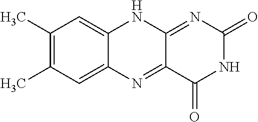 [SMILES: Cc1cc2nc3c(=O)[nH]c(=O)nc-3[nH]c2cc1C] | 4 |
| 7,8-dimethylbenzo[g]pteridine-2,4(3H,10H)-dione | |
| 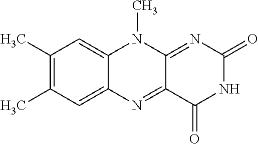 [SMILES: Cc1cc2nc3c(=O)[nH]c(=O)nc-3n(C)c2cc1C] | 5 |
| 7,8,10-trimethylbenzo[g]pteridine-2,4(3H,10H)-dione | |
| 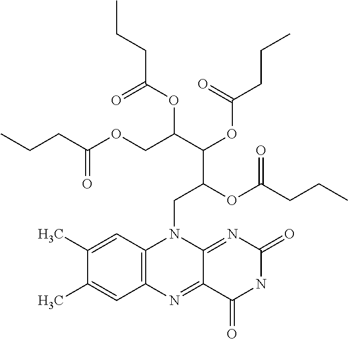 [SMILES: CCCC(=O)OCC(OC(=O)CCC)C(OC(=O)CCC)C(Cn1c2nc(=O)[nH]c(=O)c-2nc2cc(C)c(C)cc21)OC(=O)CCC] | 6 |
| Riboflavin tetrabutyrate | |
| CAS # 752-56-7 | |
| 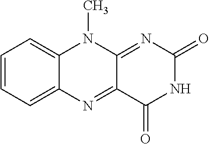 [SMILES: Cn1c2nc(=O)[nH]c(=O)c-2nc2ccccc21] | 7 |
| 10-methylbenzo[g]pteridine-2,4(3H,10H)-dione | |
| CAS # 4074-58-2 | |

and also the organic or mineral acid or base salts thereof, the optical isomers and tautomers thereof, and the solvates thereof such as hydrates, in particular the salt thereof with an alkali metal such as sodium.

According to a particular embodiment, composition (B) comprises one or more flavin derivatives as defined previously, in an amount inclusively between 0.01% and 30% by weight relative to the total weight of composition (B), in particular between 0.05% and 20%, more particularly between 0.1% and 10%, preferentially between 1% and 5% by weight relative to the total weight of composition (B).

### ii) The Polyamino Acids

Compositions (A) or (B) of the invention may comprise ii) one or more polyamino acids.

According to a particular embodiment of the invention, the polyamino acid(s) have a molecular weight of greater than or equal to 1 kilodalton (kDa), preferably a molecular weight of greater than 2 kDa, more particularly greater than 10 kDa, or even greater than 20 kDa.

In particular, the polyamino acid(s) of the invention are chosen from cytoskeleton proteins such as keratins, in particular α-keratins such as those derived from wool, fur, claws and hair, and β-keratins found in reptilian scales and claws, in their shells and in bird feathers, beaks and claws, and in porcupine needles.

Mention may be made of the following articles for further reference: Wang, Bin (2016). “Keratin: Structure, mechanical properties, occurrence in biological organisms, and efforts at bioinspiration”. Progress in Materials Science. 76: 229-318. doi:10.1016/j.pmatsci.2015.06.001 & Keratins: The human keratins: biology and pathology, Histochem Cell Biol. (2008); doi: 10.1007/s00418-008-0435-6.

Synthetic versions of keratins may also be used in the invention and are known to those skilled in the art. An example that may be mentioned is patent U.S. Pat. No. 4,722,895 A.

According to a preferred embodiment, the polyamino acid(s) are extracellular matrix proteins such as collagens. Many different types of collagen and of hydrolysed collagens are commercially available from suppliers such as Sigma-Aldrich (examples of commercial references from Sigma-Aldrich are C7661, 5162, C9791).

Preferably, the polyamino acid(s) of the invention include an amino acid sequence with glycine units (Gly-X-Y)n and comprise one or more Gly-Pro-Hyp triplets.

Synthetic versions of collagens are also known and may be used in the invention. The scientific literature describes such synthetic versions of collagens. For example, mention may be made of the articles Adv. Exp. Med. Biol.; 611: xci-xcviii (2009) and J. Am. Chem. Soc. 132, 13957-13959 (2010).

Examples of polyamino acids of the invention that may also be mentioned include polypeptides derived from silk proteins such as those mentioned in patent U.S. Pat. No. 5,747,015.

Synthetic polyamino acids may also be used in the invention. These polyamino acids may be homopolymers based on only one amino acid, in particular of formula (Ill) as defined below, preferably chosen from poly-L-proline, poly-L-hydroxyproline, poly-L-threonine, polyglycine, poly-L-serine, poly-DL-alanine, poly-L-glutamine and poly-L-tyrosine, or the synthetic polyamino acids may be copolymers based on more than one amino acid. The scientific literature describes such synthetic polyamino acids.

By way of example, mention may be made of the articles “Synthesis and Chemical Properties of Poly-α-Amino Acids”, Advances in Protein Chemistry, volume 13, 243-492, (1958), “Contribution of poly(amino acids) to advances in pharmaceutical biotechnology”, Curr. Pharm. Biotechnol. Oct; 4(5): 323-30 (2003); “Microbial degradation of poly(amino acids)”. Biomacromolecules. Jul-Aug; 5(4): 1166-76 (2004); “Occurrence, functions and biosynthesis of polyamides in microorganisms and biotechnological production”. Naturwissenschaften. Jan; 89(1): 11-22 (2002).

Natural polyamino acids based on homopolymers may also be used in the invention, such as poly-gamma-glutamic acid and poly(epsilon-L-lysine).

Natural polyamino acid-g-amino acids may also be used in the invention, such as cyanophycin composed of an aspartic acid backbone and arginine side groups.

The polyamino acids may originate from numerous sources such as keratins (wool, bodily hair, etc.), silk proteins, collagens, protamines and plant proteins (soybean, oat, wheat, etc. protein). They may also originate from keratin extracts or hydrolysates such as gelatin.

According to a particular embodiment, the polyamino acids may be chosen from collagens.

According to a particular embodiment, compositions (A) or (B) comprise one or more polyamino acids as defined previously, in an amount inclusively between 0.01% and 20% by weight relative to the total weight of composition (A) or (B), in particular between 0.05% and 10%, more particularly between 0.1% and 5%, preferentially between 0.2% and 2.5%, more preferentially between 0.25% and 1% by weight relative to the total weight of composition (A) or (B).

iii) The amines

Composition (B) comprises i) one or more compounds of formula (I) as defined previously, ii) one or more polyamino acids and iii) one or more amines other than i) the compounds of formula (I), and other than ii) the polyamino acids.

According to one variant, composition (A) comprises iii) one or more amines other than i) the compounds of formula (I), and other than ii) the polyamino acids.

According to the invention, the term “amines” means any organic molecule comprising at least one primary, secondary or tertiary amine group, other than i) the compounds of formula (I), and other than ii) the polyamino acids. The amines may be aromatic or non-aromatic, saturated or unsaturated, and natural or synthetic.

According to a preferred embodiment, the amine(s) of the invention are present in compositions (A) or (B) in an amount of between 0.01% and 15% by weight relative to the total weight of the composition which comprises same, more particularly between 0.1% and 10%, more preferentially between 0.5% and 5% by weight relative to the total weight of the composition which comprises same.

According to a particular embodiment of the invention, composition (A) of the invention comprises at least one amine, preferably at least one amino acid, preferably at least one natural amino acid.

According to a particular embodiment, composition (B) contains one or more amines chosen from amino acids, preferably from natural amino acids.

Preferably, the amino acid(s) are chosen from the compounds of formula (III) below:

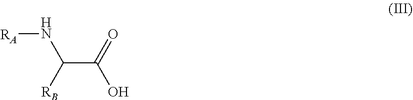

and also the organic or mineral acid or base salts thereof, the optical isomers, geometrical isomers and tautomers thereof, and the solvates thereof such as hydrates;
R_{A} represents a hydrogen atom, a (C₁-C₄)alkyl or (C₁-C₄)alkylcarbonyl group or forms, together with the radical R_{B} and the nitrogen and carbon atom that bear them, respectively, a 5- to 10-membered monocyclic or bicyclic heterocycle such as pyrrolidinyl; preferably, R_{A} represents a hydrogen atom;
R_{B} represents a hydrogen atom or a (C₁-C₆)alkyl group optionally substituted with one or more groups chosen from i) —Z—C(Z′)—Z″—R_{C} with Z, Z′, and Z″, which may be identical or different, representing an oxygen or sulfur atom, and N(RD), R_{C} and R_{D}, which may be identical or different, representing a hydrogen atom or a (C₁-C₄)alkyl group; preferably, Z, Z′ and Z″ represent a group N(R_{D}) such as NH; ii) (hetero)aryl such as imidazolyl, indolyl, phenyl, optionally substituted especially with a hydroxyl group; iii) (di)(C₁-C₄)(alkyl)amino, iv) —C(Z′)—Z″—R_{C}, with Z′, Z″ and R_{C} as defined above, in particular Z′ represents an oxygen atom, Z″ represents an oxygen atom or a group N(RD) such as NH, and R_{c} represents a hydrogen atom; v)—Z″′—R_{C}, with Z′″ representing an oxygen, sulfur or selenium atom or an NH group and R_{C} is as defined previously; in particular, RB represents a (C₁-C₆)alkyl group optionally substituted with i) —Z—C(Z′)—Z″—R_{C}, with Z, Z′, and Z″, which may be identical or different, representing an oxygen or sulfur atom, and N(R_{D}), R_{C} and R_{D}, which may be identical or different, representing a hydrogen atom or a (C₁-C₄)alkyl group; preferably Z, Z′ and Z″ represent a group N(RD) such as NH;
   or R_{A} and R_{B} form, together with the nitrogen atom which bears RA and with the carbon atom which bears R_{B}, a saturated 5- or 6-membered, preferably 5-membered, heterocycle such as a pyrrolidine ring.
   Said amino acids of formula (III) may be of L (levorotatory) or D (dextrorotatory) configuration or in a mixture, preferably of L configuration.

In particular, the amino acid(s) are chosen from alanine, arginine and proline, preferably L-alanine, L-arginine and L-proline, in particular L-arginine or L-proline, more preferentially L-arginine.

According to a preferred embodiment, the amino acid(s) of formula (III) of the invention are present in composition (A) or in composition (B) in an amount of between 0.01% and 15% by weight relative to the total weight of the composition which comprises same, more particularly between 0.05% and 10%, more preferentially between 0.1% and 5% by weight relative to the total weight of the composition which comprises same.

According to another particular embodiment of the invention, composition (A) or (B) of the invention comprises at least one amine comprising an optionally substituted (hetero)aryl group, in particular of formula (IV):

CYC-(ALK)ₚ-NRₑR_{f}

and also the organic or mineral acid salts thereof, the optical isomers thereof, and the solvates thereof such as hydrates;
in which formula (IV):
CYC represents an optionally substituted heterocycle, cycloalkyl, aryl or heteroaryl group, preferably aryl such as phenyl,
ALK represents a (C₁-C₆)alkylene group,
Rₑ and R_{f}, which may be identical or different, represent a hydrogen atom or a (C₁-C₆)alkyl group; and
p is 0 or 1.

Preferably, the compounds of formula (IV) are a benzylamine.

According to a preferred embodiment, the amine(s) of formula (IV) are present in composition (A) or in composition (B) in an amount of between 0.01% and 20% by weight relative to the total weight of the composition which comprises same, more particularly between 0.05% and 10%, more preferentially between 0.05% and 5% by weight relative to the total weight of the composition which comprises same.

The Alcohols

According to a particular embodiment, composition (A) or composition (B) as defined previously contains at least one alcohol.

According to a particular embodiment of the invention, compositions (A) or (B) of the invention comprise at least one alcohol comprising an optionally substituted (hetero)aryl group, in particular of formula (IV′):

CYC-(X)_{q}—(ALK)ₚ-OH (IV′)

and also the organic or mineral acid or base salts thereof, the optical isomers thereof, and the solvates thereof such as hydrates;
in which formula (IV′):
CYC represents a heterocycle, cycloalkyl, aryl or heteroaryl group which is optionally substituted, especially with one or more (C₁-C₄)alkyl or (di)(C₁-C₄)(alkyl)amino groups, preferably aryl such as phenyl or cycloalkyl such as cyclohexyl,
ALK represents a (C₁-C₆)alkylene group,
p and q, which may be identical or different, are equal to 0 or 1, it being understood that when p is equal to 0, then q is equal to 0 and that p is equal to 1 when q is equal to 1.

Preferably, the compounds of formula (IV′) are chosen from benzyl alcohol, phenoxyethanol and N,N-dimethylaminobenzyl alcohol.

According to another preferred embodiment of the invention, the alcohols are chosen from terpenes, more preferentially chosen from menthol, isoborneol and neomenthol.

According to another more particular embodiment, the alcohols are chosen from linear or branched, saturated or unsaturated (C₁-C₆) alkanols, such as isopropanol.

According to another more particular embodiment of the invention, composition (A) or (B) of the invention comprises at least one alcohol comprising an optionally substituted (hetero)aryl group, in particular of formula (IV″):

RₑR_{f}N-(ALK′)ₚ-CYC-(ALK)ₚ-OH

and also the organic or mineral acid or base salts thereof, the optical isomers thereof, and the solvates thereof such as hydrates;
in which formula (IV):
CYC represents an optionally substituted heterocycle, cycloalkyl, aryl or heteroaryl group, preferably aryl such as phenyl,
ALK and ALK′ independently represent a (C₁-C₆)alkylene group,
Rₑ and R_{f}, which may be identical or different, represent a hydrogen atom or a (C₁-C₆)alkyl group such as methyl; and
p and p′ are independently equal to 0 or 1.
Preferably the compounds of formula (IV″) are chosen from an N,N-dimethylaminobenzyl alcohol.

According to a preferred embodiment, the alcohol(s), in particular the alcohol(s) of formula (IV′) or (IV″), are present in compositions (A) or (B) in an amount of between 0.01% and 20% by weight relative to the total weight of the composition which comprises same, more particularly between 0.05% and 10%, more preferentially between 0.05% and 5% by weight relative to the total weight of the composition which comprises same.

According to a preferred embodiment of the invention, compositions (A) or (B) comprise one or more amines, in particular amino acids of formula (III), and one or more alcohols as defined previously, in an amount inclusively between 0.01% and 30% by weight relative to the total weight of composition (A) or (B), in particular between 0.05% and 20%, more particularly between 0.1% and 10%, preferentially between 1% and 5% by weight relative to the total weight of compositions (A) or (B).

### Step 2)—the Step of Supplying Energy by Light Radiation

According to the process of the invention, step 2) comprises the exposure of the hair to at least one artificial and/or natural light radiation which may be continuous or non-continuous, before, during or after application of compositions (A) or (B) as defined previously, to said hair.

According to the process of the invention, step 2) in particular comprises the exposure of the hair to at least one artificial and/or natural light radiation which may be continuous or non-continuous, before, during or after application of compositions (A) or (B) as defined previously, to said hair.

For the purposes of the invention, the term “artificial light radiation” means light radiation other than natural daylight (generated by the sun). In other words, natural daylight (generated by the sun) is not artificial light radiation.

The term “natural light radiation” means radiation whose sole light source is the daylight generated by the sun.

Preferably, the exposure of the hair to artificial light radiation is performed at a wavelength of between 360 and 600 nm, preferably between 375 and 550 nm, more preferentially between 400 and 480 nm.

Preferably, the artificial light radiation has an amount of energy per unit area of greater than or equal to 1 J/cm², more preferentially strictly greater than 1 J/cm², even more preferentially between 1.001 and 100 J/cm², better still between 2 and 50 J/cm², particularly preferably between 3 and 10 J/cm².

Preferably, the artificial light radiation is generated using a device chosen from arc lamps such as xenon lamps and mercury lamps, fluorescent lamps, incandescent lamps such as halogen lamps, light-emitting diodes (LEDs), organic light-emitting diodes (OLEDs) and lasers.

Mention may be made, for example, of goLITE BLU products from the company Philips, the lamp Energylight HF 3319/01 from the company Philips, the lamps Dayvia White and Messa from the company Solvital, the lamp Lumino Plus from the company Lanaform, the lamp Medibeam from the company Medibeam, the lamp M-LED 01 from the company Meimed, the lamp Lifemax Light Pod from the company Lifemax, the lamp Lite-Pad from the company Reicorp, the lamps Omnilux Clear-U and New-U from the company Omnilux, the 1000 W xenon arc lamp from the company Lot-Oriel and the lamp Camag Box 3 (4×8 W) from the company Camag.

Preferably, the duration of exposure of the hair to artificial or natural light radiation is greater than or equal to 5 seconds, more preferentially between 10 seconds and 60 minutes, even more preferentially between 15 seconds and 50 minutes, and better still between 20 seconds and 45 minutes.

According to a variant of the invention, the invention relates to a hair treatment process comprising:

1) a step of applying a composition (A) comprising:
i) one or more compounds of formula (I) as defined previously; and
ii) one or more polyamino acids as defined previously;

2) a step of supplying energy to said hair, which consists in exposing the hair to at least one artificial or natural light radiation as defined previously, steps 1) and 2) possibly being performed simultaneously or separately, preferably simultaneously or else separately 1) and then 2).

According to a particular embodiment of the invention, the hair treatment process of the invention involves at the same time (simultaneously) steps 1) and 2).

According to one variant, at the same time as the application of composition (A) to the hair, said hair is exposed to at least one artificial and/or natural light radiation.

According to a particular embodiment of the invention, when steps 1) and 2) of the process are performed simultaneously, the duration of exposure of the hair to at least one artificial and/or natural light radiation is between 5 seconds and 3 hours, preferably between 1 minute and 1 hour, more preferentially between 5 minutes and 30 minutes, such as 20 minutes; optionally followed by a step of rinsing said hair.

According to a preferred embodiment of the invention, when step 2) is performed after step 1), the time of exposure of the hair to artificial or natural light radiation is between 5 seconds and 3 hours, preferably between 1 minute and 1 hour, more preferentially between 5 minutes and 30 minutes, such as 20 minutes.

According to a variant of the invention, when step 2) of the process is performed after step 1), an intermediate leave-on step may follow step 1) before performing step 2).

The step of leaving composition (A) or (B) on the hair takes place for a time of between 5 seconds and 3 hours, preferably between 1 minute and 1 hour, such as 20 minutes, this leave-on step taking place between step 1) and step 2); optionally, a step of rinsing the hair is performed after step 2).

According to another preferred embodiment of the process, when step 2) is performed after step 1), the duration of exposure of the hair to artificial or natural light radiation is between 5 seconds and 3 hours, preferably between 1 minute and 1 hour, such as 20 minutes; said process optionally comprising a step of leaving composition (A) or (B) on the hair for a time of between 5 seconds and 3 hours, preferably between 1 minute and 1 hour, such as 15 minutes, which takes place between step 1) and step 2); a step of rinsing the hair is performed after step 1).

According to a variant of the invention, the invention relates to a hair treatment process comprising:
1. a step of applying a composition (B) comprising:
   a. one or more compounds of formula (I) as defined previously;
   b. one or more identical or different amines of formula (Ill) as defined previously;
   c. one or more polyamino acids as defined previously;
   d. optionally one or more alcohols as defined previously, preferably chosen from the alcohols of formulae (IV′) and/or (IV″);
1i) optionally followed by a leave-on time as defined previously;
2. a step of supplying energy to said hair, which consists in exposing the hair to at least one artificial or natural light radiation as defined previously;
2i) optionally followed by a step of rinsing the hair, steps 1) and 2) possibly being performed simultaneously or separately, preferably simultaneously or else separately 1) and then 2), or 1), then 1i), then 2) and then optionally 2i).

According to a particular embodiment of the invention, the hair treatment process of the invention using composition (B) involves at the same time (simultaneously) steps 1) and 2).

According to one variant, at the same time as the application of composition (B) to the hair, said hair is exposed to at least one artificial and/or natural light radiation.

According to a particular embodiment of the invention, when steps 1) and 2) of the process using composition (B) are performed simultaneously, the duration of exposure of the hair to at least one artificial and/or natural light radiation is between 5 seconds and 3 hours, preferably between 1 minute and 1 hour, such as 20 minutes; optionally followed by a step of rinsing the hair.

According to a preferred embodiment of the invention using composition (B), when step 2) is performed after step 1), the duration of exposure of the hair to artificial or natural light radiation is between 5 seconds and 3 hours, preferably between 1 minute and 1 hour, such as 15 minutes, and the process optionally includes a step of leaving composition (B) on the hair for a time of between 5 seconds and 3 hours, preferably between 1 minute and 1 hour, such as 20 minutes, this leave-on step taking place between step 1) and step 2), the process optionally comprising a step of rinsing the hair performed after step 2).

According to another preferred embodiment of the invention using composition (B), when step 2) is performed after step 1), the duration of exposure of the hair to artificial or natural light radiation is between 5 seconds and 3 hours, preferably between 1 minute and 1 hour, such as 20 minutes; and the process optionally comprises a step of leaving composition (B) on the hair for a time of between 5 seconds and 3 hours, preferably between 1 minute and 1 hour, such as 15 minutes, this leave-on step taking place between step 1) and step 2); the process optionally comprising a step of rinsing the hair performed after step 1).

According to a variant of the invention, the invention relates to a hair treatment process comprising:

1) a step of applying a composition (A) comprising:
a. one or more compounds of formula (I) as defined previously;
b. optionally one or more identical or different amines preferably chosen from the amines of formula (III) and/or (IV) as defined previously, preferably chosen from the amines of formula (III);
c. one or more polyamino acids as defined previously;
d. optionally one or more alcohols as defined previously, preferably chosen from the alcohols of formula (IV′) and/or (IV″):

1i) optionally followed by a leave-on time as defined previously;

2) a step of supplying energy to said hair, which consists in exposing the hair to at least one artificial or natural light radiation as defined previously,

2i) optionally followed by a step of rinsing the hair;

steps 1) and 2) possibly being performed simultaneously or separately, preferably simultaneously or else separately 1) and then 2), or 1), then 1i), then 2) and then optionally 2i).

Compositions (A) or (B) of the invention may be applied to dry or wet hair, preferably to dry hair.

The bath ratio of the applied compositions may range from 0.1 to 10, more particularly from 0.2 to 5 and preferably between 0.5 and 3. The term “bath ratio” means the ratio between the total weight of the applied composition and the total weight of hair to be treated.

In particular, the step of applying composition (A) or (B) to the hair may be followed by a leave-on time. The leave-on time, namely the time of contact of composition (A) or (B) on the hair, is preferably at least 5 minutes, preferably between 10 and 60 minutes and preferably between 15 and 45 minutes.

Rinsing of the hair may optionally be envisaged after applying composition (A) or (B) and optionally after the leave-on time.

The hair may then optionally be wrung dry, preferably wrung dry.

Hair Treatment Process

According to one variant, the process of the invention is performed on damaged, bleached or sensitized hair.

According to a particular form of the invention, the process of the invention is a hair treatment process comprising:

1) a step of applying to wet or dry hair a composition (A) as defined previously; and then
1i) optionally a step of leaving the composition on the hair, preferably for at least 1 minute, preferably for a time of between 5 and 60 minutes, more preferentially between 10 and 45 minutes; and then
1ii) optionally wringing out, and then
1iii) optionally drying in the open air or with a heating device which delivers heat of less than or equal to 80° C., for example using a hood or a hairdryer, and then
2) a step of supplying energy to said hair, which consists in exposing the hair to at least one artificial or natural light radiation; and then
2i) a rinsing step.

According to a particular form of the invention, the process of the invention is a hair treatment process comprising:

1) a step of applying to wet or dry hair a composition (B) as defined previously; and then
1i) optionally a step of leaving the composition on the hair, preferably for at least 1 minute, preferably for a time of between 5 and 60 minutes, more preferentially between 10 and 45 minutes; and then
1ii) optionally wringing out, and then
1iii) optionally drying in the open air or with a heating device which delivers heat of less than or equal to 80° C., for example using a hood or a hairdryer, and then
2) a step of supplying energy to said hair, which consists in exposing the hair to at least one artificial or natural light radiation; and then
2i) a rinsing step.

According to another variant, the process is performed on natural hair, i.e. hair that has not undergone any chemical treatment such as dyeing, bleaching or permanent-waving.

Compositions (A) or (B)

The Compositions

The composition(s) of the invention are cosmetic, i.e. they contain a physiologically acceptable medium, that is to say a medium that is compatible with human keratin materials such as the skin (of the body, face, around the eyes or the scalp), the hair, the eyelashes, the eyebrows, bodily hair, the nails or the lips.

The physiologically acceptable medium of the composition(s) used in the process according to the invention is advantageously an aqueous medium. It may be constituted, for example, of water or of a mixture of water and of at least one cosmetically acceptable organic solvent. Examples of organic solvents that may be mentioned include C₂-C₄ lower alcohols, such as ethanol and isopropanol; polyols, especially those containing from 2 to 6 carbon atoms, for instance glycerol, propylene glycol, butylene glycol, pentylene glycol, hexylene glycol, dipropylene glycol or diethylene glycol; polyol ethers, for instance 2-butoxyethanol, propylene glycol monomethyl ether and diethylene glycol monomethyl ether or monoethyl ether; and mixtures thereof.

The cosmetic compositions of the invention are preferably water-based and then comprise water at a concentration ranging from 5% to 99.9%, better still from 30% to 99% and even better still from 50% to 98% by weight relative to the total weight of the composition, and even more preferentially from 60% to 98% by weight relative to the total weight of the composition.

The composition used according to the invention may also contain one or more cosmetic additives chosen from nonionic, anionic, cationic and amphoteric surfactants, vitamins and provitamins other than the compounds of formula (I) including panthenol, fillers, dyestuffs, nacreous agents, opacifiers, sequestrants, film-forming polymers, cationic, anionic or neutral polymers, associative polymers, plasticizers, silicones, thickeners, oils, antifoams, moisturizers, emollients, penetrants, fragrances and preserving agents; preferably one or more nonionic, anionic, cationic or amphoteric surfactants, cationic, anionic or neutral polymers, or associative polymers.

Depending on their nature and the purpose of the composition, the usual cosmetic ingredients may be present in usual amounts, which can be readily determined by those skilled in the art and which may be, for each ingredient, between 0.01% and 80% by weight. Those skilled in the art will take care to select the ingredients included in the composition, and also the amounts thereof, so that they do not harm the properties of the compositions of the present invention.

These compositions may be packaged in pump-action bottles or in aerosol containers, so as to apply the composition in vaporized (lacquer) form or in the form of a mousse. Such packaging forms are indicated, for example, when it is desired to obtain a spray or a mousse, for treating the hair. In these cases, the composition preferably comprises at least one propellant.

pH of the compositions:

The pH of compositions (A) or (B) is preferably between 3 and 10, more particularly in the region of neutrality between 6 and 8, such as 7.

The pH values may be adjusted with an organic or mineral acid, or with an alkaline agent chosen from mineral or organic or hybrid alkaline agents or mixtures thereof.

The term “organic acid” means an acid, i.e. a compound that is capable of releasing a cation or proton H⁺ or H₃O⁺, in aqueous medium, which includes at least one optionally unsaturated, linear or branched C₁-C₂₀ hydrocarbon-based chain, a (hetero)cycloalkyl or (hetero)aryl group and at least one acidic chemical function chosen in particular from carboxyl C(O)OH, sulfonic SO₃H, sulfinic SO₂H, phosphonic POSH and phosphinic PO₂H₂.

More particularly, the acids used are chosen from hydrochloric acid HCl, hydrobromic acid HBr, sulfuric acid H₂SO₄, alkylsulfonic acids: (C₁-C₆)Alk-S(O)₂OH such as methylsulfonic acid and ethylsulfonic acid; arylsulfonic acids: Ar—S(O)₂OH such as benzenesulfonic acid and toluenesulfonic acid; (C₁-C₆)alkoxysulfinic acids: Alk—O—S(O)OH such as methoxysulfinic acid and ethoxysulfinic acid; aryloxysulfinic acids such as tolueneoxysulfinic acid and phenoxysulfinic acid; phosphoric acid H₃PO₄; triflic acid CF₃SO₃H and tetrafluoroboric acid HBF₄, and carboxylic acid(s) of formula (D) below:

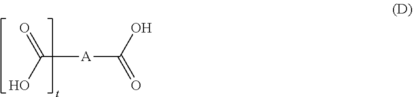

in which formula (D) or a salt thereof:
A represents a saturated or unsaturated, cyclic or non-cyclic, and aromatic or non-aromatic hydrocarbon-based group, which is monovalent when t is 0 or polyvalent when t is greater than or equal to 1, comprising from 1 to 50 carbon atoms, which is optionally interrupted with one or more heteroatoms and/or optionally substituted, especially with one or more hydroxyl groups; preferably, A represents a monovalent (C₁-C₆)alkyl group or a polyvalent (C₁-C₆)alkylene group optionally substituted with one or more hydroxyl groups.

Particularly, the carboxylic acid(s) of formula (D) as defined previously, and preferably the acid(s) used, are alpha-hydroxy acids such as lactic acids, glycolic acids, tartaric acids or citric acids.

The mineral alkaline agent(s) are preferably chosen from ammonia, alkaline carbonates or bicarbonates such as sodium or potassium carbonates and sodium or potassium bicarbonates, sodium hydroxide or potassium hydroxide, or mixtures thereof.

According to an advantageous embodiment of the invention, the alkaline agent(s) are organic amines, i.e. they contain at least one substituted or unsubstituted amino group.

The organic alkaline agent(s) are more preferentially chosen from organic amines with a pK_{b} at 25° C. of less than 12, preferably of less than 10 and even more advantageously of less than 6. It should be noted that it is the pK_{b} corresponding to the function of highest basicity.

Hybrid compounds that may be mentioned include the salts of the amines mentioned previously with acids such as carbonic acid or hydrochloric acid. The organic alkaline agent(s) are chosen, for example, from alkanolamines, oxyethylenated and/or oxypropylenated ethylenediamines, amino acids and the compounds of formula (E) below:

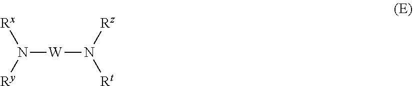

in which formula (E):
W is a divalent C₁-C₆ alkylene radical optionally substituted with a hydroxyl group or a C₁-C₆ alkyl radical, and/or optionally interrupted with one or more heteroatoms such as oxygen or NR^{u};
R^{x}, R^{y}, R^{z}, R^{t} and R^{u}, which may be identical or different, represent a hydrogen atom or a C₁-C₆ alkyl, C₁-C₆ hydroxyalkyl or C₁-C₆ aminoalkyl radical.

Preferably, the alkanolamine is ethanolamine (or monoethanolamine).

In one variant of the invention, the composition comprises, as alkaline agent, one or more alkanolamines (preferably ethanolamine) and aqueous ammonia. In this variant, the alkanolamine(s) are present in a predominant amount relative to the aqueous ammonia.

The examples that follow serve to illustrate the invention without, however, being limiting in nature.

### EXAMPLES

### 1^{st} Series of Tests

The compositions below were prepared in the following amounts in g% comprising riboflavin 5′-monophosphate sodium salt hydrate, L-arginine and water buffered at pH 7.

### Sample Preparation

Samples of hair (type II Caucasian, 2.7 g, 27 cm long) were shampooed, combed and air-dried before use. The shampoo washing protocol consists in wetting the hair with water, applying 0.4 g of shampoo (sodium lauryl sulfate at 2% in water) per gram of hair, massaging the lock from the root to the end between two fingers ten times, rinsing by 15 passes with water at 37° C. and then draining dry between two sheets.

5.4 g of compositions 1 to 8 were applied to each lock of hair and spread uniformly, massaging the hair between the fingers. The locks treated with compositions 1 to 4 were left horizontally on aluminium foil for 15 minutes and then irradiated using a 1000 W xenon arc lamp from the company Lot-Oriel (irradiation of 20 minutes on the exposed side of the lock, then the lock is turned over and irradiated for 20 minutes, i.e. an irradiation of 40 minutes for the entire lock). The locks were then shampooed by means of the above protocol, combed and air-dried.

A series of locks treated with compositions 5 to 8 was prepared in an identical manner except for the irradiation step. Instead of the irradiation step, locks 5 to 8 were placed in the dark on aluminium foil for 40 minutes, before being washed, combed and air-dried.

Processes

| | | | Riboflavin 5′- | |
|---|---|---|---|---|
| | | | monophosphate | |
| | Water/pH = | L- | sodium salt | Irradi- |
| Compositions | 7 buffer | Arginine | hydrate | ation |
| 1 | qs | — | — | YES |
| (comparative/ | | | | |
| placebo) | | | | |
| 2 | qs | 2 g % | — | YES |
| (comparative) | | | | |
| 3 | qs | — | 10 g % | YES |
| (invention) | | | | |
| 4 | qs | 2 g % | 10 g % | YES |
| (invention) | | | | |
| 5 | qs | — | — | NO |
| (comparative/ | | | | |
| placebo) | | | | |
| 6 | qs | 2 g % | — | NO |
| (comparative) | | | | |
| 7 | qs | — | 10 g % | NO |
| (comparative) | | | | |
| 8 | qs | 2 g % | 10 g % | NO |
| (comparative) | | | | |

### Evaluation of the Volume and Frizziness of the Hair

All the locks were placed in a glovebox (closed box) with a relative humidity regulated at 80%. Photographs were taken immediately (at T₀) and after 3 hours (T3h) at 80% and evaluated by comparing the shape of the locks before and after and relative to the untreated lock (placebo, lock 1).
The volume/frizziness is evaluated by measuring the mid-height width of the locks. The smaller the width at t0, the better the control of the hair volume. The smaller the difference between the mid-height width of the lock subjected to the high hygrometry for 3 hours and the mid-height width at T₀, the better the frizziness control at high hygrometry.

### Results

### Table of Results

| | Mid-height width | Mid-height width | |
|---|---|---|---|
| Lock treated with | of the lock at | of the lock at | Δ T3 h − T0 |
| composition No. | T0 (mm) | T3 h (mm) | (mm) |
| 1 | 55 | 82 | 29 |
| 2 | 53 | 83 | 30 |
| 3 | 36 | 51 | 15* |
| (invention) | | | |
| 4 | 37 | 56 | 19* |
| (invention) | | | |
| 5 | 52 | 80 | 28 |
| 6 | 51 | 81 | 30 |
| 7 | 39 | 78 | 39 |
| 8 | 37 | 82 | 45 |

*= significantly favourable result (<20 mm)

The results show that locks 3 and 4 according to the invention have: smaller mid-height widths at T0 (after irradiation) than the locks outside the invention, which means that the process of the invention gives the hair better volume control.
In addition, a significantly smaller difference is seen for processes 3 and 4 according to the invention between the mid-height width of the locks subjected to high hygrometry for 3 hours and the mid-height width at T0, which means better volume (frizziness) control at high hygrometry when compared with the differences of comparative examples 1, 2 and 5 to 8.
A second study was performed modifying the concentration of the molecules. The locks were prepared according to the same experimental protocol as for the preceding processes 1 to 8.

Processes

| | | | Riboflavin 5′- | |
|---|---|---|---|---|
| | | | monophosphate | |
| | Water/pH = | L- | sodium salt | Irradi- |
| Compositions | 7 buffer | Arginine | hydrate | ation |
| 9 | qs | 2 g % | 0.5 g % | YES |
| 10 | qs | 2 g % | 1.0 g % | YES |

### Table of Results

| | Mid-height width | Mid-height width | |
|---|---|---|---|
| Process of | of the lock at | of the lock at | Δ T3 h-T0 |
| the invention | T0 (mm) | T3 h (mm) | (mm) |
| 9 | 38 | 49 | 11 |
| 10 | 37 | 54 | 17 |

It is seen from the results in the table that the process of the invention makes it possible to obtain very satisfactory Δ T3h−T0 (mm) results since they are less than 20 mm.

### 2^{nd} Series of Tests

### Preparation of the Compositions

| Ingredient | Supplier |
|---|---|
| Riboflavin 5′-monophosphate | Sigma Aldrich - Reference: F6750 |
| sodium salt hydrate | |
| L-Arginine | Sigma Aldrich - Reference: A5006 |
| Collagen | Sigma Aldrich - Reference: C7661 |
| Water at pH 7 | Dulbeco - Reference: 14190-094 |

### Preparation of the Locks

| | g % in the formulations | | | | |
|---|---|---|---|---|---|
| | Riboflavin 5′- | | | | |
| | monophosphate | | | | |
| | sodium salt | L- | Collagen | Water | Irradi- |
| Lock No. | hydrate | Arginine | 3 | at pH 7 | ation |
| 11 | — | — | — | — | Yes |
| 12 | — | — | — | — | No |
| 13 | — | — | — | qs 100 | Yes |
| 14 | — | — | — | qs 100 | No |
| 15 | — | — | 0.5 | qs 100 | Yes |
| 16 | — | — | 0.5 | qs 100 | No |
| 17 | — | 2 | — | qs 100 | Yes |
| 18 | — | 2 | — | qs 100 | No |
| 19 | 0.5 | — | — | qs 100 | Yes |
| 20 | 0.5 | | | qs 100 | No |
| 21 | 0.5 | 2 | 0.5 | qs 100 | Yes |
| (invention) | | | | | |
| 22 | 0.5 | 2 | 0.5 | qs 100 | No |

2 g of formulations 11 to 22 were applied to locks of dry hair (2.7 g, 20 cm long, hair damaged by bleaching to have an alkaline solubility (AS) value of 20).
Locks 11, 13, 15, 17, 19 and 21 were combed and then coated with a transparent plastic film to limit the water evaporation, they were left to stand for 15 minutes and then irradiated for 20 minutes (turning the lock over after 10 minutes to irradiate the underside). The plastic film was removed, followed by rinsing thoroughly with tap water at 37° C. for 15 seconds; the locks were shampooed with a 2% lauryl sulfate solution (0.4 g of shampoo/g of hair) while massaging the locks from the root to the end between two fingers 10 times, and then rinsed thoroughly with tap water for 15 seconds, drained between two sheets of absorbent paper, combed and then dried. This shampooing protocol consisting in rinsing, shampooing, rinsing, draining, combing and then drying was repeated four times.
Locks 12, 14, 16, 18, 20 and 22 were combed and then coated with a transparent plastic film to limit the water evaporation, and then wrapped in aluminium foil to protect them from light, and then left to stand in the absence of light for 35 minutes. The aluminium foil and the plastic film were removed and the locks were rinsed thoroughly with tap water at 37° C. for 15 seconds, and then shampooed with a 2% lauryl sulfate solution (0.4 g of shampoo/g of hair) while massaging the locks from the root to the end between two fingers 10 times, rinsed thoroughly with tap water for 15 seconds, drained between two sheets of absorbent paper, combed and then dried. This shampooing protocol consisting in rinsing, shampooing, rinsing, draining, combing and then drying was repeated four times.

### Evaluation

### Root Lifting Test

The locks were held vertically with the root downward and the distance between the root and the highest point of the lock was measured.
The highest point of the lock is the highest point of the lock before it curves to fall by the effect of gravity.
The capacity of a hair treatment to make the lock remain vertical indicates the root-lifting capacity of the treatment and thus the capacity of the treatment to give the head of hair volume.
The root lifting is calculated using the formula:

% lifting (cm)=100×(initial lifting (t0h) in cm of treated lock−initial lifting (t0h) in cm of untreated lock).

The higher the value of the “% lifting”, the greater the root lifting.

### Humidity Resistance

All the locks held vertically by the root were placed in a glovebox (closed box) with a relative humidity regulated at 80%. The distance between the root and the highest point of the lock was measured immediately (at T0) and after 3 hours at 80% relative humidity (T3h).
The smaller the difference between T0 and T3h, the greater the humidity resistance and the better the volume control of the hair at high hygrometry.
The greater the difference between T0 and T3h, the poorer the humidity resistance, which characterizes the absence of volume control of the hair at high hygrometry.
If the distance between the root and the highest point of the lock after 3 hours has not changed relative to the initial state T0, then the treatment is judged to be resistant to high hygrometry and thus satisfactory.

### Results

i) Lifting of the Root

| Lock | | |
|---|---|---|
| No. | Lifting at T0 (cm) | % Lifting of the root |
| 11 | 1.0 | 0 |
| 12 | 1.0 | 0 |
| 13 | 1.0 | 0 |
| 14 | 1.0 | 0 |
| 15 | 1.1 | 10 |
| 16 | 1.1 | 10 |
| 17 | 1.0 | 0 |
| 18 | 1.0 | 0 |
| 19 | 1.0 | 0 |
| 20 | 1.0 | 0 |
| 21 | 1.4 | 40 |
| 22 | 1.1 | 10 |

ii) Humidity Resistance Test
   Only the examples which showed lifting of the root were evaluated.

| | Lifting at T0 | Lifting at T3 h | |
|---|---|---|---|
| Lock No. | (cm) | (cm) | Difference (cm) |
| 15 | 1.1 | 0.8 | 0.3 |
| 16 | 1.1 | 0.8 | 0.3 |
| 21 | 1.4 | 1.4 | 0 |
| 22 | 1.1 | 0.8 | 0.3 |

### Conclusion

Lock 21 of the invention shows very good root lifting and also very good humidity resistance.
Lock 21 of the invention was washed five times (as for all the other locks) before undergoing the evaluation (root lifting test and humidity resistance), which demonstrates that treatment 21 of the invention is also resistant to repeated washing, such as five shampoo washes.

### 3^{rd} Series of Tests

Comparative Study (vs. WO2016/126121)
The use of the same yeast (Saccharomyces cerevisiae) as described in WO2016/126121 is not possible since the details of the supplier are not given neither any procedure to prepare the yeast nor the extraction procedure of the riboflavin. After a literature search it was concluded that the concentration of riboflavin in S. cerevisiae is between 0.8-1.5 μM. Thus the higher concentration of 1.5 μM has been used to do the comparative study. Therefore, a composition comprising 20% by weight of S. cerevisiae comprises an equivalent quantity of riboflavin of 1.13 g×10⁻⁴% by weight. A particular reference for the cytosolic concentration of riboflavin in S. cerevisiae is Biosynthesis of Vitamin B2 (Riboflavin), Annual Review of Nutrition, Vol. 20:153-167 (Volume publication date July 2000)—https://doi.org/10.1146/annurev.nutr.20.1.153
In this study, poly-L-tyrosine has been used as a polyaminoacide:

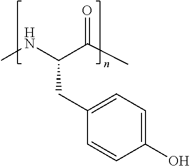

The following formulations were prepared. The ingredients were mixed, stirred for one hour in a closed amber vial then cooled to room temperature before use.

| Formu- | | | | | |
|---|---|---|---|---|---|
| lation | | | | Poly-L- | Qsp 5% Triton |
| number | Riboflavin¹ | | Collagen² | tyrosine³ | X-100⁴ in Water |
| 1 | 0.1 | g | — | | 10 g |
| 2 | 1.13 × 10⁻⁵ | g | — | | 10 g |
| 3 | 0.1 | g | 0.2 g | | 10 g |
| 4 | 1.13 × 10⁻⁵ | g | 0.2 g | | 10 g |
| 5 | 0.1 | g | | 0.2 g | 10 g |
| 6 | 1.13 × 10⁻⁵ | g | | 0.2 g | 10 g |

¹Sigma Aldrich - Ref. R7649
²Sigma Aldrich - Ref. 5162
³Sigma Aldrich - Ref. P1800
⁴Sigma Aldrich - Ref. X-100

### Preparation of Hair Swatches

| Example | Formulation number | Irradiation |
|---|---|---|
| 1 | 1 | Natural light |
| (Invention) | | |
| 2 | 1 | Artificial light |
| (Invention) | | |
| 3 | 2 | Natural light |
| (Comparative) | | |
| 4 | 3 | Natural light |
| (Invention) | | |
| 5 | 3 | Artificial light |
| (Invention) | | |
| 6 | 4 | Natural light |
| (Comparative) | | |
| 7 | 5 | Natural light |
| (Invention) | | |
| 8 | 5 | Artificial light |
| (Invention) | | |
| 9 | 6 | Natural light |
| (Comparative) | | |

The hair swatches (2.7 g, 27 cm length, solubilité alcaline (SA) de 40) were placed on a piece of aluminium foil. 5 g of each formulation was applied onto each hair swatch. The swatches were covered with a transparent plastic film to reduce evaporation. Examples 1, 3, 4, 6, 7 and 9 were exposed to natural light for 30 minutes. After 15 of the 30 minutes the swatches were turned over to ensure each swatch was exposed to at least 15 minutes of natural light. The irradiance of natural light was measured at 64 microW/cm2 using a Thor PM100D Optical Power Meter.
Examples 2, 5 and 8 were irradiated with a home-made array of LED lights emitting light at 450 nm for 30 minutes. The array of LEDs were positioned 5 cm above the swatch and the irradiance measured at 26 mW/cm2). After 15 of the 30 minutes of irradiation the swatches were turned over to ensure each side of the swatch was irradiated to at least 15 minutes.
After exposure to the light the swatches were suspended vertically, covered with a towel and left to dry.

### Shampooing

The swatches were subjected to 5 repeat washes. Washing consisted of placing the swatch in a bath containing 100mL of 3% sodium lauryl ether sulfate (SLES) for 1 minute then rinsing for 10 seconds under tap water. The process is repeated 4 times using fresh SLES solution each time. At the end of the fifth wash and rinse the swatches were dried in a professional helmet hairdryer at 60° C.

### Hair Repair Tests

### Anti-frizz Test:

The swatches were hung vertically in a humidity controlled glove box. The width of the swatches was measured at half height of the swatch (measure at to). The humidity was increased to 80% and maintained at 80% for 3 hours. The width of the swatches was measured at half height of the swatch (measure at t=3 h). The difference between the two measurements indicates the ability to reduce frizz and thus indicates that the fibre has been repaired.

| | Width of | Width of | |
|---|---|---|---|
| | swatch at t₀ | swatch at | |
| Example | (mm) | t = 3 h (mm) | Δ3 h-t₀ (mm) |
| 1 | 33 | 52 | 19 |
| (Invention) | | | |
| 2 | 31 | 44 | 13 |
| (Invention) | | | |
| 3 | 36 | 69 | 33 |
| (Comparative) | | | |

The results show that example 2 (riboflavin at 1% with artificial light irradiation) has a better performance than example 1 which in turn has a much better performance than example 3 (riboflavin at 1.13 g×10⁻⁴% and natural light irradiation).

### Anti-breakage Test:

The swatches were suspended vertically and combed 30 times with a fine toothed comb at a speed of 30 cm/sec. The amount of broken hair was collected and weighed. The lower the weight of hair collected the better the active is at reducing the breakage of hair, i.e. repairing the hair.

| Example | Irradiation | Amount of broken hair (g) |
|---|---|---|
| 4 | Natural light | 0.0562 |
| (Invention) | | |
| 5 | Artificial light | 0.0365 |
| (Invention) | | |
| 6 | Natural light | 0.1956 |
| (Comparative) | | |
| 7 | Natural light | 0.0838 |
| (Invention) | | |
| 8 | Artificial light | 0.0433 |
| (Invention) | | |
| 9 | Natural light | 0.1812 |
| (Comparative) | | |

The results show that examples 5 and 8 using 1% riboflavin and artificial light irradiation have a better performance than examples 4 and 7 using 1% riboflavin and natural light irradiation which in turn has a better performance than examples 6 and 9 (riboflavin at 1.13 g×10⁻⁴% and natural light irradiation)

1-21. (canceled)

22. A method for cosmetic hair treatment wherein the method comprises:
1) applying to the hair a composition (A) comprising i) at least one flavin derivative; and
2) supplying energy to the hair by exposing the hair to at least one artificial or natural light radiation;
wherein the at least one flavin derivative is chosen from compounds of formula (I) below: 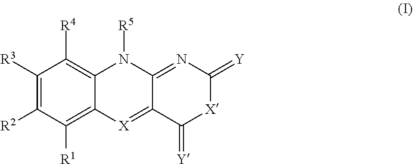
organic or mineral acid or base salts thereof, optical isomers thereof, geometrical isomers thereof, tautomers thereof, solvates thereof, or mixtures thereof;
wherein in formula (I):
R¹, R², R³ and R⁴, which may be identical or different, are chosen from a hydrogen atom, a halogen atom, or a group chosen from hydroxyl, (C₁-C₆)alkyl, (C₁-C₆)alkoxy, (C₁-C₆)alkylthio, (di)(C₁-C₆)(alkyl)amino, or nitro(so);
R⁵ is chosen from a hydrogen atom or a (C₁-C₈)alkyl group optionally substituted with at least one group chosen from i) hydroxyl, ii) R⁶—C(Y″)—O—, wherein R⁶ is chosen from a hydrogen atom, a (C₁-C₄)alkyl group, or an aryl(C₁-C₄)alkyl group, and Y″ is chosen from an oxygen or sulfur atom, or N(R⁷), wherein R⁷ is chosen from a hydrogen atom or a (C₁-C₄)alkyl group, iii) phosphoric (HO)2P(O)—O—, iv) —O—[(HO)P(O)—O]ₙ-Suc-Het, wherein Suc represents a divalent sugar group, Het represents a heteroaryl group, and n is an integer equal to 0, 1 or 2;
X is chosen from a nitrogen atom or a methylene group C(R⁸), wherein R⁸ is chosen from a hydrogen atom or a (C₁-C₄)alkyl group;
X′ is chosen from an oxygen atom or a group NR⁹, wherein R⁹ is chosen from a hydrogen atom or a (C₁-C₆)alkyl group; and
Y and Y′, which may be identical or different, are chosen from an oxygen or sulfur atom, or a group NR¹⁰, wherein R¹° is chosen from a hydrogen atom or a (C₁-C₆)alkyl group;
wherein the at least one flavin derivatives is present in an amount ranging from 0.01% to 30% by weight, relative to the total weight of the composition (A); and
wherein steps 1) and 2) are performed simultaneously or step 2) is performed after step 1).

23. The method of claim 22, wherein in formula (I), R¹ and R⁴ represent a hydrogen atom and R² and R³ are chosen from a hydrogen atom or a (C₁-C₄)alkyl group.

24. The method of claim 22, wherein in formula (I), R⁵ is chosen from a (C₁-C₆)alkyl group substituted with at least one group, which may be identical or different, chosen from: i) hydroxyl, ii) R⁶—C(O)—O—, wherein R⁶ is chosen from a hydrogen atom or a (C₁-C₄)alkyl group; iii) phosphoric (HO)2P(O)—O—, iv) —O—[(HO)P(O)—O]ₙ-Suc-Het, wherein Suc represents a monosaccharide group, Het represents an optionally substituted heteroaryl group, and n is an integer equal to 1 or 2.

25. The method of claim 22, wherein the Suc is chosen from a divalent monosaccharide radical comprising a heterocycle including 4 or 5 carbon atoms and having the following formula: 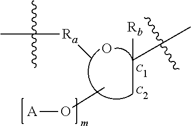
wherein,
Rₐ is chosen from a (C₁-C₄)alkylene group and is linked to the rest of the divalent monosaccharide radical via at least one phosphate group, wherein Rₐ is in position C⁵ if Suc is in pyranose form or Rₐ is in position C⁴ if Suc is in a furanose form;
R_{b} is chosen from a hydrogen atom or a group —CH₂—O—A;
A is chosen from a hydrogen atom, a (C₁-C₆)alkyl group, a hydroxy-function-protecting group, or a (C₁-C₄)alkyl group, wherein when m is greater than or equal to 2 and at least two groups A—O are contiguous, the at least two groups A—O optionally together form a linear or branched (C₁-C₆)alkylene chain;
m is equal to 1, 2 or 3; and
symbol [symbol] represents two substitution parts of the divalent monosaccharide

26. The method of claim 22, wherein in formula (I), R⁵ represents the following group: 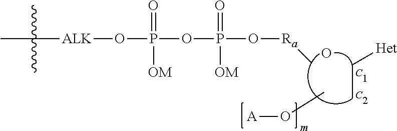
wherein:
Rₐ is chosen from a (C₁-C₄)alkylene group and is linked to the rest of R⁵ via at least one phosphate group, wherein Rₐ is in position C⁵ if Suc is in pyranose form or Rₐ is in position C⁴ if Suc is in furanose form;
A is chosen from a hydrogen atom, a (C₁-C₆)alkyl group, a hydroxy-function-protecting group, or a (C₁-C₄)alkyl group, wherein when m is greater than or equal to 2 and at least two groups A—O are contiguous, the at least two groups A—O optionally together form a linear or branched (C₁-C₆)alkylene chain;
Het is an optionally substituted heteroaryl group;
m is equal to 1, 2 or 3;
ALK is chosen from a (C₁-C₆)alkylene group optionally substituted with at least one hydroxyl group;
M represents a hydrogen atom or a cationic counterion; and
symbol [symbol] represents a part that is linked to the rest of the R⁵.

27. The method of claim 26 , wherein Het is chosen from a purine or pyrimidine nitrogenous base.

28. The method of claim 22, wherein in formula (I), X is a nitrogen atom;
X′ is a group NR⁹, wherein R⁹ is chosen from a hydrogen atom or a (C₁-C₆)alkyl group;
and Y and Y′ are identical and chosen from an oxygen atom or a sulfur atom.

29. The method of claim 22, wherein the at least one compound of the compounds of formula (I) is chosen from compounds 1 to 7 below:
(2R,3S,4S)-5-(7,8-dimethyl-2,4-dioxo-3,4 dihydrobenzo[g]pteridin-10(2H)-yl)-2,3,4-trihydroxypentyl dihydrogen phosphate, or riboflavin 5′-phosphate or riboflavin-5′-phosphate (compound 1);
7,8-dimethyl-10-((2R,3R,4S)-2,3,4,5-tetrahydroxypentyl)benzo[g]pteridine-2,4-(3H,10H)-dione, or vitamin B2, lactoflavin (compound 2);
Flavin adenine dinucleotide (FAD) (compound 3);
7,8-dimethylbenzo[g]pteridine-2,4(3H,10H)-dione (compound 4);
7,8,10-trimethylbenzo[g]pteridine-2,4(3H,10H)-dione (compound 5);
Riboflavin tetrabutyrate (compound 6); or
10-methylbenzo[g]pteridine-2,4(3H,10H)-dione (compound 7);

| Structure | |
|---|---|
| 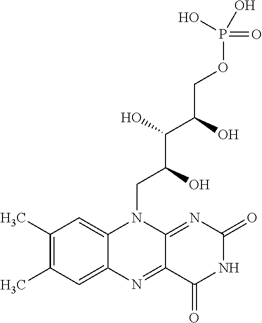 [SMILES: Cc1cc2nc3c(=O)[nH]c(=O)nc-3n(C[C@H](O)[C@H](O)[C@H](O)COP(=O)(O)O)c2cc1C] | 1 |
| 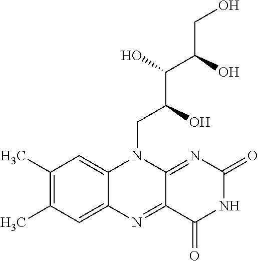 [SMILES: Cc1cc2nc3c(=O)[nH]c(=O)nc-3n(C[C@H](O)[C@H](O)[C@H](O)CO)c2cc1C] | 2 |
| 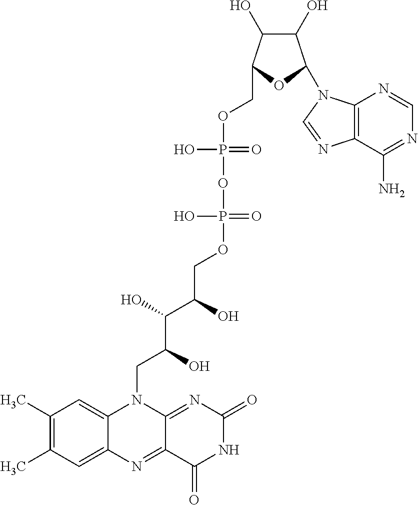 | 3 |
| 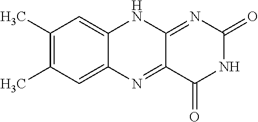 [SMILES: Cc1cc2nc3c(=O)[nH]c(=O)nc-3[nH]c2cc1C] | 4 |
| 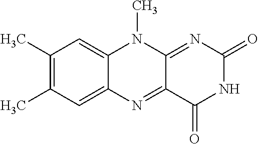 [SMILES: Cc1cc2nc3c(=O)[nH]c(=O)nc-3n(C)c2cc1C] | 5 |
| 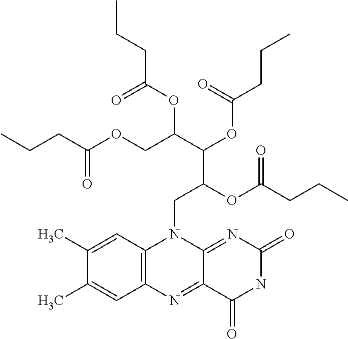 [SMILES: CCCC(=O)OCC(OC(=O)CCC)C(OC(=O)CCC)C(Cn1c2nc(=O)[nH]c(=O)c-2nc2cc(C)c(C)cc21)OC(=O)CCC] | 6 |
| 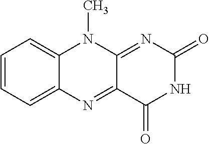 [SMILES: Cn1c2nc(=O)[nH]c(=O)c-2nc2ccccc21] | 7 |

organic or mineral acid or base salts thereof, optical isomers thereof, tautomers thereof, solvates thereof, or mixtures thereof.

30. The method of claim 22, wherein the composition (A) further comprises ii) at least one polyamino acid.

31. The method of claim 30, wherein the at least one polyamino acid has a molecular weight of greater than or equal to 1 kilodalton (kDa).

32. The method of claim 30, wherein the at least one polyamino acid is chosen from:
cytoskeleton proteins;
synthetic keratins;
polyamino acids comprising an amino acid sequence with glycine units (Gly-X-Y)n and at least one Gly-Pro-Hyp triplet;
polyamino acids derived from silk or from silk proteins;
synthetic polyamino acids;
natural polyamino acids based on homopolymers;
natural polyamino acid-g-amino acids; or
mixtures thereof.

33. The method of claim 30, wherein the at least one polyamino acid is present in an amount ranging from 0.01% to 20% by weight, relative to the total weight of the composition (A).

34. The method of claim 30, wherein the composition (A) further comprises iii) at least one amine other than i) the at least one compound of formula (I), and other than ii) the at least one polyamino acid; and wherein the at least one amine is chosen from aromatic, non-aromatic, saturated, unsaturated, oligomeric, polymeric, natural, or synthetic amines.

35. The method of claim 34, wherein the at least one amine is an amino acid, chosen from compounds of formula (III) below: 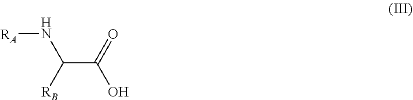
organic or mineral acid or base salts thereof, optical isomers thereof, geometrical isomers thereof, tautomers thereof, solvates thereof, or mixtures thereof;
wherein:
R_{A} is chosen from a hydrogen atom, a (C₁-C₄)alkyl group, or a (C₁-C₄)alkylcarbonyl group, or forms, together with a radical R_{B}, and the nitrogen atom and the carbon atom that bear R_{A} and R_{B}, respectively, a 5- to 10-membered monocyclic or bicyclic heterocycle; and
R_{B} is chosen from a hydrogen atom or a (C₁-C₆)alkyl group optionally substituted with at least one group chosen from i) —Z—C(Z′)—Z″—R_{C}, wherein Z, Z′, and Z″, which may be identical or different, are chosen from an oxygen atom, a sulfur atom, or N(R_{D}), and wherein R_{C} and R_{D}, which may be identical or different, are chosen from a hydrogen atom or a (C₁-C₄)alkyl group; ii) (hetero)aryl, optionally substituted with a hydroxyl group; iii) (di)(C₁-C₄)(alkyl)amino, iv) —C(Z′)—Z″—R_{C}, wherein Z′, Z″, which may be identical or different, are chosen from an oxygen atom, a sulfur atom, or N(RD), and wherein R_{C} and R_{D}, which may be identical or different, are chosen from a hydrogen atom or a (C₁-C₄)alkyl group; v) —Z″—R_{C}, wherein Z″′ is chosen from an oxygen atom, a sulfur atom, a selenium atom, or an NH group, and R_{C} is chosen from a hydrogen atom or a (C₁-C₄)alkyl group.

36. The method of claim 22, wherein , the step 2) comprises exposing the hair to artificial light radiation with a wavelength of between 360 and 600 nm; and wherein the artificial light radiation is generated using a device chosen from arc lamps, fluorescent lamps, incandescent lamps, light-emitting diodes (LEDs), organic light-emitting diodes (OLEDs), or lasers.

37. The method of claim 22, wherein in step 2), the duration of exposing the hair to the artificial or natural light radiation is greater than or equal to 5 seconds.

38. The method of claim 22, wherein when step 2) is performed after step 1); optionally, a step of leaving the composition (A) on the hair for a period of time ranging from 5 seconds to 3 hours takes place between step 1) and step 2); and optionally, a step of rinsing the hair is performed after step 2).

39. A composition (B) comprising i) at least one compound chosen from compounds of formula (I) below; ii) at least one polyamino acid, and iii) at least one amine other than the compounds of formula (I) and the at least one polyamino acid; 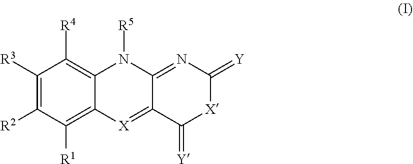
wherein in formula (I):
R¹, R², R³ and R⁴, which may be identical or different, are chosen from a hydrogen atom, a halogen atom, or a group chosen from hydroxyl, (C₁-C₆)alkyl, (C₁-C₆)alkoxy, (C₁-C₆)alkylthio, (di)(C₁-C₆)(alkyl)amino, or nitro(so);
R⁵ is chosen from a hydrogen atom or a (C₁-C₈)alkyl group optionally substituted with at least one group chosen from i) hydroxyl, ii) R⁶—C(Y″)—O—, wherein R⁶ is chosen from a hydrogen atom, a (C₁-C₄)alkyl group, or an aryl(C₁-C₄)alkyl group, and Y″ is chosen from an oxygen or sulfur atom, or N(R⁷), wherein R⁷ is chosen from a hydrogen atom or a (C₁-C₄)alkyl group, iii) phosphoric (HO)₂P(O)—O—, iv) —O—[(HO)P(O)—O]ₙ-Suc-Het, wherein Suc represents a divalent sugar group, Het represents a heteroaryl group, and n is an integer equal to 0, 1, or 2;
X is chosen from a nitrogen atom or a methylene group C(R⁸), wherein R⁸ is chosen from a hydrogen atom or a (C₁-C₄)alkyl group;
X′ is chosen from an oxygen atom or a group NR⁹, wherein R⁹ is chosen from a hydrogen atom or a (C₁-C₆)alkyl group; and
Y and Y′, which may be identical or different, are chosen from an oxygen atom, a sulfur atom, or a group NR¹⁰, wherein R¹⁰ is chosen from a hydrogen atom or a (C₁-C₆)alkyl group.

40. A multi-compartment device comprising:
at least one compartment containing:
a first ingredient i) comprising at least one compound of formula (I) below, 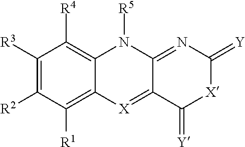
optionally a second ingredient ii) comprising at least one polyamino acid and optionally a third ingredient iii) comprising at least one amine other than the at least one compound of formula (I) and other than the at least one polyamino acid; and
a separate compartment comprising an artificial-light-emitting device providing an artificial light radiation with a wavelength ranging from 360 nm to 600 nm;
wherein the first ingredient i), the second ingredient ii), and the third ingredient iii) are contained together in one compartment or separately in two or three compartments; and
wherein in formula (I):
R¹, R², R³ and R⁴, which may be identical or different, are chosen from a hydrogen atom, a halogen atom, or a group chosen from hydroxyl, (C₁-C₆)alkyl, (C₁-C₆)alkoxy, (C₁-C₆)alkylthio, (di)(C₁-C₆)(alkyl)amino, or nitro(so);
R⁵ is chosen from a hydrogen atom or a (C₁-C₈)alkyl group optionally substituted with at least one group chosen from i) hydroxyl, ii) R⁶—C(Y″)—O—, wherein R⁶ is chosen from a hydrogen atom, a (C₁-C₄)alkyl group, or an aryl(C₁-C₄)alkyl group, and Y″ is chosen from an oxygen atom, a sulfur atom, or N(R⁷), wherein R⁷ is chosen from a hydrogen atom or a (C₁-C₄)alkyl group, iii) phosphoric (HO)₂P(O)—O—, iv) —O—[(HO)P(O)—O]ₙ-Suc-Het, wherein Suc represents a divalent sugar group, Het represents a heteroaryl group, and n is an integer equal to 0, 1 or 2;
X is chosen from a nitrogen atom or a methylene group C(R⁸), wherein R⁸ is chosen from a hydrogen atom or a (C₁-C₄)alkyl group;
X′ is chosen from an oxygen atom or a group NR⁹, wherein R⁹ is chosen from a hydrogen atom or a (C₁-C₆)alkyl group; and
Y and Y′, which may be identical or different, are chosen from an oxygen atom, a sulfur atom, or a group NR¹⁰, wherein R¹° is chosen from a hydrogen atom or a (C₁-C₆)alkyl group.
